(19) United States
(12) Patent Application Publication
(10) Pub. No.: US 2005/0123500 A1
(43) Pub. Date: Jun. 9, 2005

(54) Means for improving the appearance of mammalian hair and nails

(75) Inventors: Amy Violet Trejo, Mason, OH (US); Mark Leslie Kacher, Mason, OH (US); George C. Daher, East Windsor, NJ (US); Catherine Lowman Oppenheimer, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: THE PROCTER & GAMBLE COMPANY

(21) Appl. No.: 10/921,031

(22) Filed: Aug. 18, 2004

Related U.S. Application Data
(63) Continuation-in-part of application No. 10/769,280, filed on Jan. 30, 2004.
(60) Provisional application No. 60/444,140, filed on Jan. 31, 2003.

(51) Int. Cl. A61K 7/04; A61K 31/015; A61K 35/78

(52) U.S. Cl. 424/74; 424/766; 514/62; 514/560; 424/93.45; 514/763

(57) ABSTRACT

Improving the cosmetic appearance of mammalian hair and/or nails, especially increasing the shine of mammalian hair and decreasing the brittleness and breakage of mammalian nails. More particularly, the invention relates to improving the appearance of mammalian hair and/or nails by orally administering a dietary supplement comprising a safe and effective amount of a sugar amine to a mammal, preferably a human. In another aspect, the invention also relates to a method and an article of commerce.

### CROSS REFERENCE TO RELATED APPLICATION(S)

This application is a continuation-in-part of U.S. application Ser. No. 10/769,280, filed Jan. 30, 2004, which claims the benefit of priority to U.S. Provisional Application No. 60/444,140, filed Jan. 31, 2003, all of which are herein incorporated by reference.

### TECHNICAL FIELD

The present invention relates to improving the cosmetic appearance of mammalian hair and/or nails, particularly increasing the shine of mammalian hair and decreasing the breakage of mammalian nails. More particularly, the invention relates to improving the appearance of mammalian hair and/or nails by orally administering a dietary supplement comprising a safe and effective amount of a sugar amine to a mammal, preferably a human.

### BACKGROUND

Mammalian hair and nails are subject to insults by many extrinsic and intrinsic factors. Extrinsic factors include environmental pollution, wind, heat, low humidity, ultraviolet radiation (e.g., from sun exposure), and the like. Intrinsic factors include chronological aging and other biochemical changes produced by the body. Additionally, diet and nutrition can have a major impact on the appearance of the hair and nails. In today's society, many people's diets are nutrient deficient and/or imbalanced, resulting in less than optimal hair and nail health and appearance. These extrinsic and intrinsic factors, as well as an insufficient diet, can contribute to hair appearance that is lifeless and dull, rather than attractive and shiny, and to unattractive nails that are brittle and break easily.

Thus, many consumers have hair that is not as visually attractive, and in particular not as shiny, as desired. In addition, many consumers have nails that are not as attractive, and in particular broken, brittle, or not as strong as desired. Accordingly, there is a need to provide consumers with a means of achieving a shinier hair appearance and/or stronger, unbroken, more attractive nails.

### SUMMARY OF THE INVENTION

The present invention provides consumers with a means of achieving a more attractive, shinier hair appearance and/or stronger, unbroken, more attractive nails. In particular, the invention provides for the use of a dietary supplement by a mammal, preferably a human, for the purpose of increasing the appearance of hair shine and/or increasing the attractiveness of the nails by decreasing brittleness and breakage. The dietary supplement comprises a safe and effective amount of a sugar anine.

In another aspect, the present invention provides a method for informing consumers that oral administration of the dietary supplement can increase the attractiveness of the hair and/or nails. In yet another aspect, the invention is directed to an article of commerce.

All documents cited herein are incorporated by reference in their entirety; the citation of any document is not to be construed as an admission that it is prior art with respect to the present invention.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The term “safe and effective amount” as used herein means an amount of a compound or composition sufficient to significantly induce a positive benefit, preferably a positive appearance benefit of the hair and/or nails, but low enough to avoid serious side effects, i.e., to provide a reasonable benefit to risk ratio, within the scope of sound judgment of the skilled artisan.

A. Use of Dietary Supplement for the Purpose of Improving the Appearance of Hair and/or Nails

According to the present invention, the dietary supplement is suitable for oral consumption and is administered orally. In preferred embodiments, the dietary supplement is in the form of capsules (e.g., those meant to be swallowed or chewed), tablets (e.g., those meant to be swallowed or chewed), powders (e.g., the powder can be added to water, milk, or another liquid), liquids (e.g., either in ready to drink form or suitable for dilution in another beverage), or nutritional foodstuffs. For example, the dietary supplement can be in the form of nutritional bars (e.g., meal or snack bars), cookies, candies (e.g., taffies, caramels, jellies, chocolate melt-aways, chews such as fruit chews, gums), syrups, or beverages (e.g., ready to drink or in the form of concentrates or powders).

As used herein, the term “dietary supplement” is broad enough to include not only single dosage forms, but also multiple dosage forms used in conjunction with one another. For instance, in one embodiment, the dietary supplement is a capsule (e.g., containing both omega fatty acids and glucosamine). In another embodiment, the dietary supplement is two capsules (e.g., one containing omega fatty acids and one containing glucosamine). In yet another embodiment, the dietary supplement comprises a beverage containing glucosamine that is consumed in conjunction with a capsule containing omega fatty acids. As used here, “consumed in conjunction with” or “used in conjunction with” means orally consumed at the same time or at different times.

As used in the present application, the term “improving the appearance of hair and/or nails” includes, but is not limited to, increasing hair shine, luster, and/or radiance; improving and/or increasing nail strength, reducing nail brittleness and/or nail breakage; prevention and/or correction of the functional disorders of the pilo-sebaceous unit of mammals, particularly humans, for instance the treatment of oily and/or hyper-seborrheic scalp, thin hair, hyper-trichosis, and/or alopecia; increasing the density of hair, especially human hair, and/or reducing the heterogeneity of hair diameter and/or improving (e.g., increasing the rate of) the growth of hair and/or nails and/or preventing and/or reducing and/or delaying hair loss; and increasing hair volume.

As used herein, the term “heterogeneity of hair diameter” refers to a significant variation in the hair diameter in a specific region of the scalp; some hair having a physiological diameter in the range of 100 um, and others, in the nearest proximity of those hairs, having a reduced diameter (e.g., thin hair). Thus, by “reducing heterogeneity of the diameter,” it is meant increasing the diameter of thin hair. By “increasing the density,” it is meant increasing the number of hairs per square centimeter of skin or scalp.

The regimen of consumption can vary according to the form of the dietary supplement. For instance, if the dietary supplement is in the form of a capsule or tablet, it is preferably taken orally with one or more meals daily. For example, the capsule or tablet may be taken daily with breakfast, lunch, and/or dinner. More preferably, there is oral consumption of the capsule or tablet form of dietary supplements of the present invention over an extended period during the subject's lifetime, preferably three times a day for a period of at least about a week, more preferably three times a day for a period of at least about one month, even more preferably three times a day for at least about three months, even more preferably three times a day for at least about six months, and more preferably still three times a day for at least about one year. While benefits are obtainable after various maximum periods of use (e.g., five, ten or twenty years), it is preferred that oral consumption of the capsule or tablet form of dietary supplements of the present invention continues throughout the subject's lifetime.

In combination with the oral consumption of the dietary supplements of the present invention, a product suitable for topical application can optionally be applied to the hair and/or nails as part of a combined treatment regimen. In one embodiment, one or more topical products are applied to the hair and/or nails for the purpose of increasing their health and/or attractiveness, in addition to orally consuming the dietary supplement for the same purpose. In another embodiment, a combined treatment regimen is not used; rather, the dietary supplement is orally consumed but topical products are not also used for the purpose of improvement in hair and/or nail appearance (e.g., increasing hair shine and/or decreasing nail breakage and/or brittleness).

The dietary supplement herein comprises: (1) a safe and effective amount of a sugar amine, and (2) optionally, any suitable optional components.

1. Sugar Amines (Amino Sugars)

As used herein, “sugar amine” refers to an amine derivative of a six-carbon sugar. As used herein, the term “sugar amine” is broad enough to include not only sugar amines, but also pharmaceutically acceptable salts thereof. Furthermore, the terms “sugar amine” and “sugar amines” are used interchangeably to mean one sugar amine or a mixture of more than one sugar amine.

Preferably, the daily dose of sugar amine from about 0.1 g to about 3 g, more preferably from about 0.75 g to about 2.5 g, and still more preferably from about 1 g to about 2 g.

Examples of sugar amines that are useful herein include, but are not limited to, glucosamine, N-acetyl glucosamine, mannosamine, N-acetyl mannosamine, galactosamine, N-acetyl galactosamine; their derivatives; their salts; and mixtures thereof. Preferably, the sugar amine is glucosamine. Preferred glucosamines include N-acetyl glucosamine, glucosamine, and glucosamine hydrochloride. Additionally, combinations of two or more sugar amines may be used.

2. Optional Components

The dietary supplement according to the present invention can optionally include any suitable optional components. These optional components can include, but are not limited to, polyunsaturated fatty acids, anti-oxidants, vitamins, micronutrient metals, pro-biotics, pre-biotics, and combinations thereof. As used herein, the terms “optional component” and “optional components” are used interchangeably to mean one optional component or a mixture of more than one optional component.

a. Polyunsaturated Fatty Acids

The dietary supplement can include a safe and effective amount of polyunsaturated fatty acids. Polyunsaturated fatty acids are essentially polyethylenic acid (i.e., comprising at least two carbon-carbon double bonds). As used herein, the terms “polyunsaturated fatty acid” and “polyunsaturated fatty acids” are used interchangeably to mean one polyunsaturated fatty acid or a mixture of more than one polyunsaturated fatty acid. As used herein, the term “polyunsaturated fatty acids” is broad enough to include mono-, di-, and tri-glycerides, essential fatty acids, salts of fatty acids, and salts of essential fatty acids.

Preferably, the polyunsaturated fatty acids used herein are essential fatty acids. As used herein, the terms “essential fatty acid” and “essential fatty acids” are used interchangeably to mean one essential fatty acid or a mixture of more than one essential fatty acid. As used herein, “essential fatty acids” refers to fats that are essential to the diet because the body cannot produce them and are classified as either omega-3 fatty acids or omega-6 fatty acids. Preferably, the daily dose is from at least about 0.1 g to about 3 g, more preferably from about 0.75 g to about 2.5 g and, even more preferably, from about 1 g to about 2 g, of the essential fatty acid.

Examples of omega-3 fatty acids that are useful herein include alpha-linolenic acid, stearidonic acid, eicosapentanoic acid, (EPA), docosahexanoic acid (DHA), and mixtures thereof. Preferably, the omega-3 fatty acid is EPA, DHA, or mixtures thereof.

Examples of omega-6 fatty acids that are useful herein include linoleic acid, gamma-linolenic acid, arachidonic acid, and mixtures thereof. Preferably, the omega-6 fatty acid is gamma-linolenic acid.

b. Anti-Oxidants

The dietary supplement of the present invention can include a safe and effective amount of an anti-oxidant. As used herein, the terms “anti-oxidant” and “anti-oxidants” are used interchangeably to mean one anti-oxidant or a mixture of more than one anti-oxidant. Preferably, the daily dose is from about 0.0001 g to about 1 g, more preferably from about 0.0001 g to about 0.5 g and, even more preferably, from about 0.0005 g to about 0.1 g, of the anti-oxidant.

Anti-oxidants such as ester-C+, ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox^{R}), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pidolate, nordihydroguaiaretic acid, bioflavonoids, lysine, methionine, proline, superoxide dismutase, silymarin, mixed carotenoids (i.e. beta-carotene, lutein, lycopene), tea extracts, grape skin/seed extracts, melanin, and rosemary extracts may be used. Preferred anti-oxidants are selected from tocopherol acetate and other esters of tocopherol, more preferably tocopherol acetate.

c. Vitamins

The dietary supplement of the present invention can include a safe and effective amount of a vitamin. As used herein, the terms “vitamin” and “vitamins” are used interchangeably to mean one vitamin or a mixture of more than one vitamin. Furthermore, the terms “vitamin” or “vitamins” are broad enough as used herein to include pro-vitamins (e.g., beta-carotene). Preferably, the daily dose is from about 0.0001 g to about 1 g, more preferably from about 0.0001 g to about 0.5 g and, even more preferably, from about 0.0005 g to about 0.1 g, of the vitamin.

Examples of vitamins that are useful herein include vitamin A, vitamin B, vitamin C, vitamin D, vitamin E, vitamin K; their derivatives; and mixtures thereof.

a) Vitamin B

Examples of Vitamin B compounds useful herein include vitamin B₁, vitamin B₂, vitamin B₃, vitamin B₅, vitamin B₆, vitamin B₁₂, vitamin B₁₅, their derivatives, and mixtures thereof.

For Example, a safe and effective amount of a vitamin B₃ compound may be used. When vitamin B₃ compounds are used, the daily dose is preferably from about 0.00001 g to about 1 g, more preferably from about 0.0001 g to about 0.5 g and, even more preferably, from about 0.0005 g to about 0.1 g, of the vitamin B₃ compound.

As used herein, “vitamin B₃ compound” means a compound having the formula: 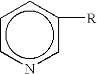
wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Examples of suitable vitamin B₃ compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

The vitamin compounds may be included as the substantially pure material, or as an extract obtained by suitable physical and/or chemical isolation from natural (e.g., plant) sources.

Also for example, a safe and effective amount of a vitamin B₆ compound may be used. The vitamin B₆ compounds useful herein include pyridoxine, esters of pyridoxine (e.g., pyridoxine tripalmitate), amines of pyridoxine (e.g., pyridoxamine), salts of pyridoxine (e.g., pyridoxine HCl) and derivatives thereof, including pyridoxamine, pyridoxal, pyridoxal phosphate, and pyridoxic acid. More preferably, the vitamin B₆ is selected from the group consisting of pyridoxine, esters of pyridoxine and salts of pyridoxine. Most preferably, the vitamin B₆ is pyridoxine HCl.

Vitamin B₆ can be synthetic or natural in origin and can be used as essentially as pure compounds or mixtures of compounds (e.g., extracts from natural sources or mixtures of synthetic materials). Vitamin B₆ is generally found in many foodstuffs, especially yeast, liver and cereals. As used herein, “vitamin B₆” includes isomers and tautomers of such and is commercially available from Sigma Chemical Co., St. Louis, Mo.

When vitamin B₆ compounds are present in the instant invention, the daily dose is preferably from about 0.0001 g to about 1 g, more preferably from about 0.0001 g to about 0.5 g and, even more preferably, from about 0.0005 g to about 0.1 g, of the vitamin B₆ compound.

d. Micronutrient Metals

The dietary supplement of the present invention may also include a micronutrient metal. As used herein, the term “micronutrient metal” and “micronutrient metals” are used interchangeably to mean one micronutrient metal or a mixture of more than one micronutrient metal. As used herein, “micronutrient metal” refers to a metal that provides nutrients to a mammalian body that are necessary for proper total nutrition. Preferably, the daily dose is from about 0.0001 g to about 1 g, more preferably from about 0.0001 g to about 0.5 g and, even more preferably, from about 0.0005 g to about 0.1 g, of micronutrient metal.

Examples of micronutrient metal that are useful herein include iron, zinc, selenium, copper, manganese; their derivatives; their salts; and mixtures thereof. Preferably, the micronutrient metal is copper, selenium, zinc and mixtures thereof.

e. Pro-biotics

The dietary supplement may also include a pro-biotic. As used herein, the terms “pro-biotic” and “pro-biotics” are used interchangeably to mean one pro-biotic or a mixture of more than one pro-biotic. As used herein, “pro-biotic” refers to an organism that contributes to the health and balance of the intestinal tract. Preferably, the daily dose is from about 0.0001 g to about 1 g, more preferably from about 0.0001 g to about 0.5 g, and even more preferably from about 0.0005 g to about 0.1 g of pro-biotic. Preferred pro-biotics can include Lactobacilli and Bifidobacteria.

f. Pre-biotics

The dietary supplement of the present invention may also include a pre-biotic. As used herein, the terms “pre-biotic” and “pre-biotics” are broad enough to include one or a mixture of more than one pre-biotics. As used herein, “pre-biotic” refers to a substrate on which bacteria (i.e., pro-biotic) feeds. Preferably, the daily dose is from about 0.0001 g to about 1 g, more preferably from about 0.0001 g to about 0.5 g and, even more preferably, from about 0.0005 g to about 0.1 g, of the pre-biotic.

Examples of pre-biotics useful herein include inulin and fructooligosaccharides and mixtures thereof.

B. Method for Improving Consumer Hair and/or Nail Appearance

In another aspect, the present invention provides a method for improving human hair and/or nail appearance. The method comprises the steps of:
(1) making available to a consumer an orally administered dietary supplement comprising a safe and effective amount of a sugar amine;
(2) communicating to said consumer that oral administration of said dietary supplement can improve the appearance of said consumer's hair or nails;
(3) acquisition of said orally administered dietary supplement by said consumer; and
(4) oral administration of said dietary supplement by said consumer.

The dietary supplement can be made available to the consumer by any suitable means. For instance, it can be offered for sale at a retail outlet or for purchase through a catalogue or via the internet. Furthermore, any suitable means of communication can be used to communicate to the consumer that oral administration of the dietary supplement can improve the appearance of the consumer's hair or nails. For example, communication can be in the form of printed material, an electronic message via the internet, or a broadcast message through television or radio. The communication can inform the consumer that oral administration of the dietary supplement can improve hair health, hair shine, hair luster, or hair radiance, or can improve nail health, nail strength, or can reduce nail brittleness or breakage.

In a particular embodiment, the communication informs the consumer that oral administration of the dietary supplement can lead to prevention and/or correction of the functional disorders of the pilo-sebaceous unit of mammals, particularly humans, and more specifically for the treatment of oily and/or hyper-seborrheic scalp, thin hair, hyper-trichosis, and/or alopecia. In another embodiment, the communication informs the consumer that oral administration of the dietary supplement can increase the density of hair, especially human hair, and/or reduce the heterogeneity of their diameter and/or improve their growth and/or prevent and/or reduce and/or delay hair loss. In still another embodiment the communication informs the consumer that oral administration of the dietary supplement can increase the hair volume.

As used herein, the term “heterogeneity of hair diameter” refers to a significant variation in the hair diameter in a specific region of the scalp; some hair having a physiological diameter in the range of 100 um, and others, in the nearest proximity of those hairs, having a reduced diameter (e.g., thin hair). Thus, by “reducing heterogeneity of the diameter,” it is meant increasing the diameter of thin hair. By “increasing the density,” it is meant increasing the number of hairs per square centimeter of skin or scalp.

The consumer can acquire the dietary supplement by any suitable means. For instance, the consumer can purchase the dietary supplement, can obtain the dietary supplement from a purchaser of the dietary supplement, can obtain the dietary supplement as a free sample, or can obtain the dietary supplement as a gift with, or in conjunction with, the purchase of another item. As used herein, the term “consumer” is broad enough to include both purchasers and potential purchasers of the dietary supplement.

C. Article of Commerce

In yet another aspect, the invention is directed to an article of commerce. In this aspect, the article of commerce comprises:
(1) an orally administered dietary supplement comprising a safe and effective amount of a sugar amine;
(2) at least one container for containing said dietary supplement; and
(3) a message associated with said dietary supplement, wherein said message informs a consumer that oral administration of said dietary supplement can improve the appearance of said consumer's hair or nails.

Suitable messages can include, but are not limited to, messages that inform the consumer that oral administration of the dietary supplement can improve hair health, hair shine, hair luster, or hair radiance, or can improve nail health, nail strength, or can reduce nail brittleness or breakage.

The message can be printed material attached directly or indirectly to the container, attached directly or indirectly near the container, or alternatively can be a printed, electronic, or broadcast message associated with the dietary supplement.

Any container from which the dietary supplement can be dispensed, presented, displayed, or stored is suitable. Suitable containers include, but are not limited to, bottles, boxes, bags, and pouches.

### EXAMPLES

### Example 1

### Dietary Supplement

The dietary supplement of this example comprises three separate capsules.

One Capsule, 1000 mg total.

The capsule is formulated according to the following:

| | |
|---|---|
| Tuna Oil Concentrate | (585 mg) |
| Omega 3 (EPA) Eicosapentaneoic Acid | 120 mg |
| Omega 3 (DHA) Docosahexaenoic Acid | 93.6 mg |
| Omega 6 | 24 mg |
| Other | 115 mg |
| Tocopherol | 2 mg |
| Other ingredients: | to balance, 1000 mg total |

Two capsules, 750 mg each of Glucosamine HCl.

### Example 2

### Article of Commerce

The dietary supplement of Example 1 is packaged in a bottle and offered for sale in a retail outlet. A television advertisement contains information that informs a consumer that oral administration of the dietary supplement can improve the shine, radiance, and luster of the consumer's hair.

### Example 2

### Article of Commerce

The dietary supplement of Example 1 is packaged in a pouch and offered for sale in a retail outlet. A print advertisement in a magazine contains information that informs a consumer that oral administration of the dietary supplement can decrease breakage and brittleness of the consumer's nails.

### Example 3

### Article of Commerce

The dietary supplement of Example 1 is packaged in a pouch and offered for sale in a retail outlet. A print advertisement in a magazine contains information that informs a consumer that oral administration of the dietary supplement can improve the appearance of both the hair and nails by increasing the shine of the consumer's hair and decreasing breakage of the consumer's nails.

All documents cited in the Detailed Description of the Invention are, in relevant part, incorporated hereby reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for improving human hair or nail appearance, wherein said method comprises the steps of:
(1) making available to a consumer an orally administered dietary supplement comprising a safe and effective amount of a sugar amine;
(2) communicating to said consumer that oral administration of said dietary supplement can improve the appearance of said consumer's hair, nails, or hair and nails;
(3) acquisition of said orally administered dietary supplement by said consumer; and
(4) oral administration of said dietary supplement by said consumer.

2. The method of claim 1, wherein said sugar amine comprises a component selected from the group consisting of derivatives of glucosamine, N-acetyl glucosamine, glucosamine hydrochloride, galactosamine, derivatives of galactosamine, N-acetyl galactosamine, mannosamine, derivatives of mannosamine, N-acetyl mannosamine, and mixtures thereof.

3. The method of claim 1, wherein said dietary supplement additionally comprises a safe and effective amount of a polyunsaturated fatty acid.

4. The method of claim 3, wherein said polyunsaturated fatty acid comprises an essential fatty acid selected from the group consisting of omega-3 fatty acids, omega-6 fatty acids, EPA, DHA, and mixtures thereof.

5. The method of claim 1, wherein said dietary supplement additionally comprises a safe and effective amount of an anti-oxidant selected from the group consisting of grape seed extract, ester-C+, beta-carotene, lycopene, lutein, vitamin E, vitamin C, their derivatives, their salts, and mixtures thereof.

6. The method of claim 1, wherein said dietary supplement additionally comprises a safe and effective amount of a probiotic.

7. An article of commerce comprising:
(1) an orally administered dietary supplement comprising a safe and effective amount of a sugar amine;
(2) a container for containing said dietary supplement; and
(3) a message associated with said dietary supplement, wherein said message informs a consumer that oral administration of said dietary supplement can improve the appearance of said consumer's hair, nails, or both hair and nails.

8. The article of claim 7, wherein said sugar amine comprises a component selected from the group consisting of derivatives of glucosamine, N-acetyl glucosamine, glucosamine hydrochloride, galactosamine, derivatives of galactosamine, N-acetyl galactosamine, mannosamine, derivatives of mannosamine, N-acetyl mannosamine, and mixtures thereof.

9. The article of claim 7, wherein said dietary supplement additionally comprises a safe and effective amount of a polyunsaturated fatty acid.

10. The article of claim 9, wherein said polyunsaturated fatty acid is selected from the group consisting of omega-3 fatty acids, omega-6 fatty acids, EPA, DHA, and mixtures thereof.

11. The article of claim 7, wherein said dietary supplement additionally comprises a safe and effective amount of an anti-oxidant selected from the group consisting of grape seed extract, ester-C+, beta-carotene, lycopene, lutein, vitamin E, vitamin C, their derivatives, their salts, and mixtures thereof.

12. The article of claim 7, wherein said dietary supplement additionally comprises a safe and effective amount of a probiotic.

13. The method of claim 1, where said communicating comprises communicating to said consumer that oral administration of said dietary supplement can increase the shine, luster, radiance, volume, density, or thickness of said consumer's hair.

14. The method of claim 1, where said communicating comprises communicating to said consumer that oral administration of said dietary supplement can decrease nail breakage or brittleness.

15. The article of claim 7, wherein said message informs a consumer that oral administration of said dietary supplement can increase the shine, luster, radiance, volume, density, or thickness of said consumer's hair.

16. The article of claim 7, wherein said message informs a consumer that oral administration of said dietary supplement can decrease nail breakage or brittleness.
